(19) United States
(12) Patent Application Publication
(10) Pub. No.: US 2007/0232691 A1
(43) Pub. Date: Oct. 4, 2007

(54) Use of ladostigil for the treatment of schizophrenia

(76) Inventors: Tamar Goren, Rehovot (IL); Eran Blaugrund, Rehovot (IL)

Correspondence Address: COOPER & DUNHAM, LLP, 1185 AVENUE OF THE AMERICAS, NEW YORK, NY, 10036, US

(21) Appl. No.: 11/729,733

(22) Filed: Mar. 28, 2007

Related U.S. Application Data
(60) Provisional application No. 60/788,560, filed on Mar. 31, 2006.

(51) Int. Cl. A61K 31/325

(52) U.S. Cl. 514/480

(57) ABSTRACT

Disclosed are methods for the treatment of schizophrenia comprising administering an amount of R(+)-6-(N-methyl, N-ethyl-carbamoyloxy)-N′-propargyl-1-aminoindan or a pharmaceutically acceptable salt thereof.

This application claims benefit of U.S. Provisional Application No. 60/788,560, filed Mar. 31, 2006, the contents of which are hereby incorporated by reference.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Schizophrenia is a common and serious disorder characterized by loss of contact with reality (psychosis), hallucinations (false perceptions), delusions (false beliefs), abnormal thinking, flattened affect (restricted range of emotions), diminished motivation, and disturbed work and social functioning (Merck Manual 17th ed. (1999), pg. 1564). The most enduring neurobiological hypothesis of schizophrenia is the dopamine (“DA”) hypothesis, which posits that the psychotic symptoms of schizophrenia result from mesolimbic DA hyperactivity (Abi-Dargham A., et al., Increased striatal dopamine transmission in schizophrenia: confirmation in a second cohort, Am. J. Psychiatry (1998), 155:761-7; Kapur S., et al., Dopamine D(2) receptors and their role in atypical antipsychotic action: still necessary and may even be sufficient, Biol. Psychiatry (2001), 50:873-83; and Weiner I., et al., Dopamine in schizophrenia: dysfunctional information processing in basal ganglia-thalamocortical split circuits, Di Chiara G (ed) Handbook of Experimental Pharmacology (2002), vol. 154/II, Dopamine in the CNS II. Springer-Verlag, Berlin, pp 417-472). Recently, however, an increasing role has been given to alteration of glutamatergic transmission, particularly at the N-methyl-D-asparate receptor (“NMDAR”) (Goff D. C., et al., The emerging role of glutamate in the pathophysiology and treatment of schizophrenia, Am. J. Psychiatry (2001) 158:1367-77; Javitt D. C., Negative schizophrenic symptomatology and the PCP (phencyclidine) model of schizophrenia, Hillside J. Clin. Psychiatry (1987) 9:12-35; Javitt D. C., Glycine modulators in schizophrenia, Curr. Opin. Investig. Drugs (2002) 3:1067-72; Jentsch J. D., et al., The neuropsychopharmacology of phencyclidine: from NMDAR hypofunction to the dopamine hypothesis of schizophrenia, Neuropsychopharmacology (1999) 20:201-25; and Laruelle, et al. 2003). A major reason for both hypotheses derives from findings that the administration of both amphetamine and NMDAR antagonists such as phencyclidine (“PCP”) and dizocilpine (“MK-801”) produce in healthy humans the entire spectrum of schizophrenia symptoms, namely, positive symptoms and negative symptoms/cognitive impairment, and exacerbate such symptoms in subjects afflicted with schizophrenia. Based on the above, two kinds of animal pharmacological models have evolved to study schizophrenia—amphetamine-based models considered to model the DA abnormality, and NMDAR antagonist-based models thought to model glutamatergic pathology. In humans, amphetamine induces only positive symptoms whereas NMDAR antagonists induce also negative symptoms/cognitive impairment of the disorder. As such, amphetamine is considered to model positive symptoms whereas NMDAR antagonists are considered to model negative symptoms/cognitive impairment. This differentiation is supported by the effects of the established and putative antipsychotic drugs (“APDs”) on amphetamine versus NMDAR-induced abnormalities: usually, the amphetamines are antagonized by both typical and atypical APDs whereas the NMDAR antagonists are antagonized by atypical but not typical APDs. In addition, the NMDAR antagonist abnormalities are sensitive to compounds enhancing NMDAR function via the glycineB site which have been shown to be beneficial against negative symptoms/cognitive impairment (Halberstadt, A. L., The phencyclidine-glutamate model of schizophrenia, Clin. Neuropharmacol. (1995), 18:237-49; Javitt 1987; Javitt and Zukin 1991; Goff D. C., et al., The emerging role of glutamate in the pathophysiology and treatment of schizophrenia, Am. J. Psychiatry (2001), 158:1367-77; Heresco-Levy, U., Glutamatergic neurotransmission modulation and the mechanisms of antipsychotic atypicality, Prog. Neuropsychopharmacol. Biol. Psychiatry (2003), 27:1113-23; Heresco-Levy U., et al., Comparative effects of glycine and -cycloserine on persistent negative symptoms in schizophrenia: a retrospective analysis, Schizophrenia Research (2004), 66:89-96; and Javitt D. C., et al., Decoding schizophrenia, Sci. Am. (2004) 290:48-55; Krystal J. H., et al., NMDAR antagonist effects, cortical glutamatergic function, and schizophrenia: toward a paradigm shift in medication development. Psychopharmacology (Berl) (2003), 169:215-33).

### SUMMARY OF THE INVENTION

The subject invention provides a method of treating a symptom of schizophrenia in a subject afflicted with schizophrenia comprising administering to the subject an amount of R(+)-6-(N-methyl, N-ethyl-carbamoyloxy)-N′-propargyl-1-aminoindan or a pharmaceutically acceptable salt thereof effective to treat the symptom of schizophrenia.

The subject invention also provides a pharmaceutical composition comprising R(+)-6-(N-methyl, N-ethyl-carbamoyloxy)-N′-propargyl-1-aminoindan or the pharmaceutically acceptable salt thereof and an agent which treats a symptom of schizophrenia.

The subject invention also provides use of R(+)-6-(N-methyl, N-ethyl-carbamoyloxy)-N′-propargyl-1-aminoindan or a pharmaceutically acceptable salt thereof in the manufacture of a medicament for the treatment of, or alleviation of a symptom of, schizophrenia.

The subject invention also provides a pharmaceutical composition for use in the treatment of, or alleviation of symptoms of, schizophrenia, which comprises an amount of R(+)-6-(N-methyl, N-ethyl-carbamoyloxy)-N′-propargyl-1-aminoindan or a pharmaceutically acceptable salt thereof and a pharmaceutically acceptable carrier.

### BRIEF DESCRIPTION OF THE DRAWINGS

FIG. 1: Full Experiment—Means and standard errors of the log times to complete licks 76-100 (after tone onset) of the preexposed (PE) and nonpreexposed (NPE) rats treated with MK-801 or saline, and pretreated with ladostigil tartrate at doses of 25 mg/kg (low) or 37.5 mg/kg (high), or vehicle. Forty preexposures and five conditioning trials were used. Ladostigil tartrate was chronically administered perorally prior to the preexposure (day 10 of administration) and conditioning (day 11 of administration) stages; MK-801 was administered intraperitoneally prior to the conditioning stage. Asterisks indicate a significant difference between the PE and NPE groups, namely, presence of latent inhibition.

FIG. 2: Low Dose Experiment—Means and standard errors of the log times to complete licks 76-100 (after tone onset) of the preexposed (PE) and nonpreexposed (NPE) rats treated with MK-801 or saline, and pretreated with ladostigil tartrate at a dose of 25 mg/kg (low) or vehicle. Forty preexposures and five conditioning trials were used. Ladostigil tartrate was chronically administered perorally prior to the preexposure (day 10 of administration) and conditioning (day 11 of administration) stages; MK-801 was administered intraperitoneally prior to the conditioning stage. Asterisks indicate a significant difference between the PE and NPE groups, namely, presence of latent inhibition.

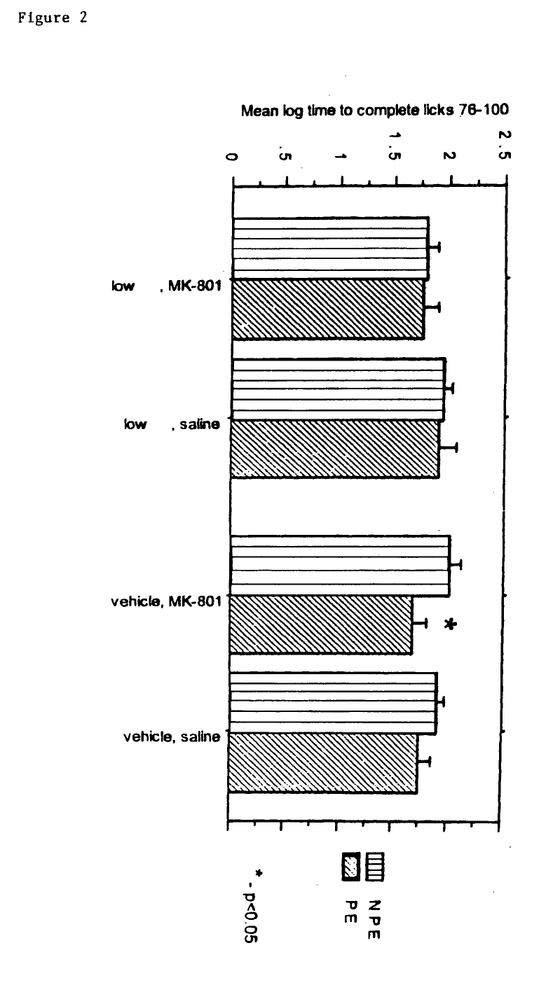

FIG. 3: High Dose Experiment—Means and standard errors of the log times to complete licks 76-100 (after tone onset) of the preexposed (PE) and nonpreexposed (NPE) rats treated with MK-801 or saline, and pretreated with ladostigil tartrate at a dose of 37.5 mg/kg (high) or vehicle. Forty preexposures and five conditioning trials were used. Ladostigil tartrate was chronically administered perorally prior to the preexposure (day 10 of administration) and conditioning (day 11 of administration) stages; MK-801 was administered intraperitoneally prior to the conditioning stage. Asterisks indicate a significant difference between the PE and NPE groups, namely, presence of latent inhibition.

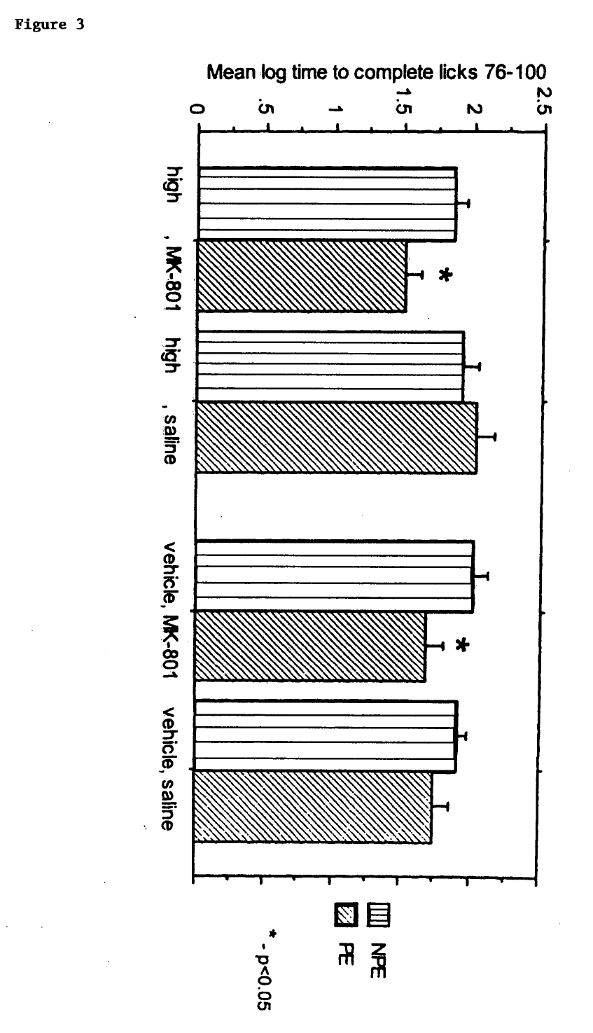

### DETAILED DESCRIPTION OF THE INVENTION

A method of treating a symptom of schizophrenia in a subject afflicted with schizophrenia comprising administering to the subject an amount of R(+)-6-(N-methyl, N-ethyl-carbamoyloxy)-N′-propargyl-1-aminoindan or a pharmaceutically acceptable salt thereof effective to treat the symptom of schizophrenia.

In an embodiment of the method, the subject is a human being.

In another embodiment of the method, the symptom of the schizophrenia is a negative symptom/cognitive impairment.

In another embodiment of the method, the administration is effected orally, parenterally, rectally or transdermally.

In another embodiment, the method comprises administering R(+)-6-(N-methyl, N-ethyl-carbamoyloxy)-N′-propargyl-1-aminoindan.

In another embodiment, the method comprises administering a pharmaceutically acceptable salt of R(+)-6-(N-methyl, N-ethyl-carbamoyloxy)-N′-propargyl-1-aminoindan.

In another embodiment of the method, the pharmaceutically acceptable salt of R(+)-6-(N-methyl, N-ethyl-carbamoyloxy)-N′-propargyl-1-aminoindan is ½ tartrate.

In another embodiment of the method, the amount of R(+)-6-(N-methyl, N-ethyl-carbamoyloxy)-N′-propargyl-1-aminoindan ½ tartrate is in the range from 0.5 mg to 2000 mg. By 0.5 mg to 2000 mg it is meant that all tenth and integer unit amounts within the range are specifically disclosed as part of the invention. Thus, 0.6, 0.7 . . . 0.9; 1, 2 . . . 1999 mg unit amounts are included as embodiments of this invention.

In yet another embodiment of the method, the amount of R(+)-6-(N-methyl, N-ethyl-carbamoyloxy)-N′-propargyl-1-aminoindan ½ tartrate is in the range from 25 mg to 105 mg. By 25 mg to 105 mg it is meant that all integer unit amounts within the range are specifically disclosed as part of the invention. Thus, 26, 27 . . . 104 mg unit amounts are included as embodiments of this invention.

In yet another embodiment of the method, the amount of R(+)-6-(N-methyl, N-ethyl-carbamoyloxy)-N′-propargyl-1-aminoindan ½ tartrate is 25 mg.

In yet another embodiment of the method, the amount of R(+)-6-(N-methyl, N-ethyl-carbamoyloxy)-N′-propargyl-1-aminoindan ½ tartrate is 8.9 mg.

In yet another embodiment of the method, the salt of R(+)-6-(N-methyl, N-ethyl-carbamoyloxy)-N′-propargyl-1-aminoindan is in crystalline form.

In a further embodiment of the method, the R(+)-6-(N-methyl, N-ethyl-carbamoyloxy)-N′-propargyl-1-aminoindan or the pharmaceutically acceptable salt thereof is in a pharmaceutical composition which also comprises at least one pharmaceutically acceptable carrier.

In yet a further embodiment of the method, the pharmaceutical composition is in the form of a tablet, capsule, pill, powder, or granule.

The subject invention also provides a pharmaceutical composition comprising R(+)-6-(N-methyl, N-ethyl-carbamoyloxy)-N′-propargyl-1-aminoindan or the pharmaceutically acceptable salt thereof and an agent which treats a symptom of schizophrenia.

In an embodiment of the composition, the agent is chlorpromazine, thioridazine, mesoridazine, trifluoperazine, fluphenazine, perphenazine, loxapine, molindone, thiothixene, haloperidol, pimozide, clozapine, risperidone, olanzapine, quetiapine, sertindole, or ziprasidone.

The subject invention also provides use of R(+)-6-(N-methyl, N-ethyl-carbamoyloxy)-N′-propargyl-1-aminoindan or a pharmaceutically acceptable salt thereof in the manufacture of a medicament for the treatment of, or alleviation of a symptom of, schizophrenia.

The subject invention also provides a pharmaceutical composition for use in the treatment of, or alleviation of symptoms of, schizophrenia, which comprises an amount of R(+)-6-(N-methyl, N-ethyl-carbamoyloxy)-N′-propargyl-1-aminoindan or a pharmaceutically acceptable salt thereof and a pharmaceutically acceptable carrier.

R(+)-6-(N-methyl,N-ethyl-carbamoyloxy)-N′-propargyl-1-aminoindan, also known as (3R)-3-(prop-2-ynylamino)-2,3,-dihydro-1H-inden-5-yl ethylmethylcarbamate, is disclosed in PCT Application Publication No. WO98/27055 (U.S. Pat. No. 6,303,650, issued Oct. 16, 2001 to Chorev), the entire contents of which are incorporated by reference. This compound has been given the nonproprietary name ladostigil.

The present invention thus provides as a compound the R(+)-enantiomer of 6-(N-methyl,N-ethyl-carbamoyloxy)-N′-propargyl-1-aminoindan and pharmaceutically acceptable salts thereof for the treatment of human patients afflicted with schizophrenia.

The present invention relates to the racemic compound and optically active isomers thereof.

The ladostigil or its salt may be prepared as pharmaceutical compositions particularly useful for the treatment of schizophrenia.

Such compositions may comprise the compound of ladostigil or pharmaceutically acceptable salts thereof, together with pharmaceutically acceptable carriers and/or excipients. In the practice of this invention, pharmaceutically acceptable salts include, but are not limited to, the mesylate, maleate, fumarate, tartrate, hydrochloride, hydrobromide, esylate, p-tolunesulfonate, benzoate, acetate, phosphate and sulfate salts. “Ladostigil tartrate” is actually R(+)-6-(N-methyl, N-ethyl-carbamoyloxy)-N′-propargyl-1-aminoindan ½ tartrate.

Such compositions may also comprise an agent which treats a symptom of schizophrenia, the agent being chlorpromazine, thioridazine, mesoridazine, trifluoperazine, fluphenazine, perphenazine, loxapine, molindone, thiothixene, haloperidol, pimozide, clozapine, risperidone, olanzapine, quetiapine, sertindole, or ziprasidone.

The compositions may be prepared as medicaments to be administered orally, parenterally, rectally or transdermally. Suitable forms for oral administration include tablets, compressed or coated pills, dragees, sachets, hard or soft gelatin capsules, sublingual tablets, syrups and suspensions; for parenteral administration the invention provides ampoules or vials that include an aqueous or non-aqueous solution or emulsion; for rectal administration there are provided suppositories with hydrophilic or hydrophobic vehicles; and for topical application as ointments and transdermal delivery there are provided suitable delivery systems as known in the art.

Specific examples of pharmaceutical acceptable carriers and excipients that may be used to formulate oral dosage forms of the present invention are described, e.g., in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

As used herein, a “pharmaceutically acceptable” carrier is one that is suitable for use with humans and/or animals without undue adverse side effects (such as toxicity, irritation, and allergic response) commensurate with a reasonable benefit/risk ratio.

As used herein, a subject “afflicted” with schizophrenia means the subject has been diagnosed with schizophrenia.

### Experimental Details

### Model

Latent inhibition (“LI”) is the process whereby pre-exposure to a stimulus retards conditioning to this stimulus when it is subsequently paired with reinforcement, and it has been used extensively to model cognitive impairments in schizophrenia. To date, LI is the only model in which amphetamine and NMDAR antagonists produce different, in fact, opposite, behavioral abnormalities, thus allowing a better screening of potential drugs, because compounds beneficial for positive symptoms and for negative symptoms produce opposite effects in the model. Briefly, amphetamine disrupts LI in rats and normal humans, and this is paralleled by disrupted LI in acute schizophrenia patients. Amphetamine-induced LI disruption is reversed by both typical and atypical APDs. In contrast, MK-801 produces abnormally persistent LI (LI present under conditions that disrupt it in normal rats) in rats, and this is paralleled by excessive LI in schizophrenia patients with predominantly negative symptoms. Consistent with the pharmacology of NMDAR antagonist models as well as with that of negative symptoms, MK-801-induced persistent LI is reversed by atypical but not typical APDs as well as by glycinergic compounds. As noted above, treatments possessing the capacity to reverse amphetamine-induced and MK-801-induced LI abnormalities must produce different and in fact opposite actions on the LI phenomenon. Drugs effective in the amphetamine model restore disrupted LI whereas drugs effective in the MK-801 model disrupt LI. Persistent LI may thus enable an accurate identification of drugs that are effective in reversing NMDAR effects and thus presumably in treating negative symptoms/cognitive impairment (Gray J. A., et al., The neuropsychology of schizophrenia, Behav. Brain Sci. (1991) 14:1-20; Moser P. C., et al., The pharmacology of latent inhibition as an animal model of schizophrenia, Brain Res. Rev. (2000), 33:275-307; and Salomon-Gaisler and Weiner 2003; Weiner I., The “two-headed” latent inhibition model of schizophrenia: modeling positive and negative symptoms and their treatment, Psychopharmacology (2003), 169:257-297).

### Effect of Ladostigil Tartrate on MK-801-Induced Persistent LI

### Apparatus and Procedure

The rats used were male Wistar rats approximately five months old, weighing 350 g to 500 g.

Rats were tested in Campden Instruments rodent test chambers with a retractable bottle. When the bottle was not present, the hole was covered by a metal lid. Licks were detected by a Campden Instruments drinkometer. The preexposed to-be-conditioned stimulus was a 10 sec, 80 dB, 2.8 kHz tone produced by a Sonalert module. Shock was supplied through the floor by a Campden Instruments shock generator and shock scrambler set at 0.5 mA and 1 sec duration. Equipment programming and data recording were computer controlled.

LI was measured in a thirst motivated conditioned emotional response (“CER”) procedure by comparing the suppression of drinking to a tone previously paired with a foot shock in rats that received non-reinforced exposure to the tone prior to conditioning (preexposed) and in rats for whom the tone was novel (non-preexposed). Parameters that do not produce LI in no-drug controls, 40 preexposures and 5 conditioning trials, were used, because persistent LI can be manifested only with such parameters.

Prior to the beginning of each LI experiment, rats were handled for about 2 min daily for 8 days. On day 9, a 23 h water restriction schedule was initiated and continued throughout the experiment. After 5 days of deprivation (days 9-13), rats were trained to drink in the experimental chambers for the next 5 days (days 14-18), for 20 min during the 1^{st} day and for 15 min per day during each of the next 4 days. Water in the test apparatus was given in addition to the daily ration of 1 h given in the home cages. The LI procedure was conducted on days 19-22 and consisted of the following stages:

### Preexposure

With the bottle removed, the preexposed (“PE”) rats received 40 tone presentations with an inter-stimulus interval of 50 sec. The nonpreexposed (“NPE”) rats were confined to the chamber for an identical period of time without receiving the tone.

### Conditioning

With the bottle removed, each rat received 5 tone-shock pairings given 5 min apart. Shock immediately followed tone termination. The first tone-shock pairing was given 5 min after the start of the session. After the last pairing, rats were left in the experimental chamber for an additional 5 min.

### Re-Baseline

Rats were given a 15 min drinking session as in initial training. Data of rats that failed to complete 600 licks were dropped from the analysis.

### Test

Each rat was placed in the chamber and allowed to drink from the bottle. When the rat completed 75 licks the tone was presented for 5 min. The following times were recorded: Time to first lick, time to complete licks 1-50, time to complete licks 51-75 (before tone onset) and time to complete licks 76-100 (after tone onset). Times to complete licks 76-100 were logarithmically transformed to allow parametric analysis of variance. Longer log times indicate stronger suppression of drinking. LI is defined as significantly shorter log times to complete licks 76-100 of the preexposed as compared to nonpreexposed rats. Data of rats that failed to complete 75 licks, thus did not reach to the tone onset, were dropped from the analysis.

### Drug Administration

On days 8-9, rats were trained to adjust to the peroral (“p.o.”) procedure by being fed with vehicle. Ladostigil tartrate (Teva Pharmaceuticals, Israel) was diluted in water and administered p.o. at doses of 37.5 mg/kg and 25 mg/kg, at a volume of 1 ml/kg. The administration began on day 10 and continued throughout the entire procedure. Rats were fed at least one hour before being put into experimental chambers, except during preexposure and conditioning days (days 19 and 20) during which rats were fed exactly two hours before being put into experimental chambers.

For comparison, the conventional acetylcholinesterase inhibitor physostigmine was also tested. Physostigmine was administered, at doses of 0.05 mg/kg and 0.15 mg/kg, 30 min before preexposure and conditioning (n per group=8).

MK-801 (dizocilpine; Merck Research Laboratories, USA) was diluted in saline and administered intraperitoneally, at a dose of 0.05 mg/kg, at a volume of 1 ml/kg, 30 min before conditioning (day 20).

No-drug controls of MK-801 and ladostigil tartrate received the corresponding vehicle.

### Experimental Design

The experiment included 12 groups in a 2×2×3 design with main factors of preexposure (PE, NPE), treatment (vehicle, MK-801) and pretreatment (vehicle, 25 mg/kg ladostigil tartrate, 37.5 mg/kg ladostigil tartrate).

### Results

TABLE 1
Determination of MAO in rat brain in LI (latent
inhibition) model for schizophrenia

| | | Brain | |
|---|---|---|---|
| | | MAO-A | MAO-B |
| | | dpm/sd | dpm/sd |
| No. of animals/group | Dose mg/kg | % inhibition | % inhibition |
| 6(1–6)/saline (dpm) | — | 9541 ± 4200 | 9490 ± 3500 |
| 6(7–12)/MK-801; water | — | 9411 ± 3160 | 8875 ± 5960 |
| 6(13–18)/saline and | 25 | 2459 ± 30174 | 1501 ± 18384 |
| ladostigil tartrate | | | |
| (dpm) | | | |
| 6(19–24)/MK-801 and | 25 | 2366 ± 29175 | 1485 ± 24183 |
| ladostigil tartrate | | | |
| (dpm) | | | |
| 6(25–30)/saline and | 37.5 | 1582 ± 27683 | 1056 ± 17189 |
| ladostigil tartrate | | | |
| (dpm) | | | |
| 6(31–36)/MK-801 and | 37.5 | 1494 ± 38784 | 1007 ± 29689 |
| ladostigil tartrate | | | |
| (dpm) | | | |

### Times to Complete Licks 76-100

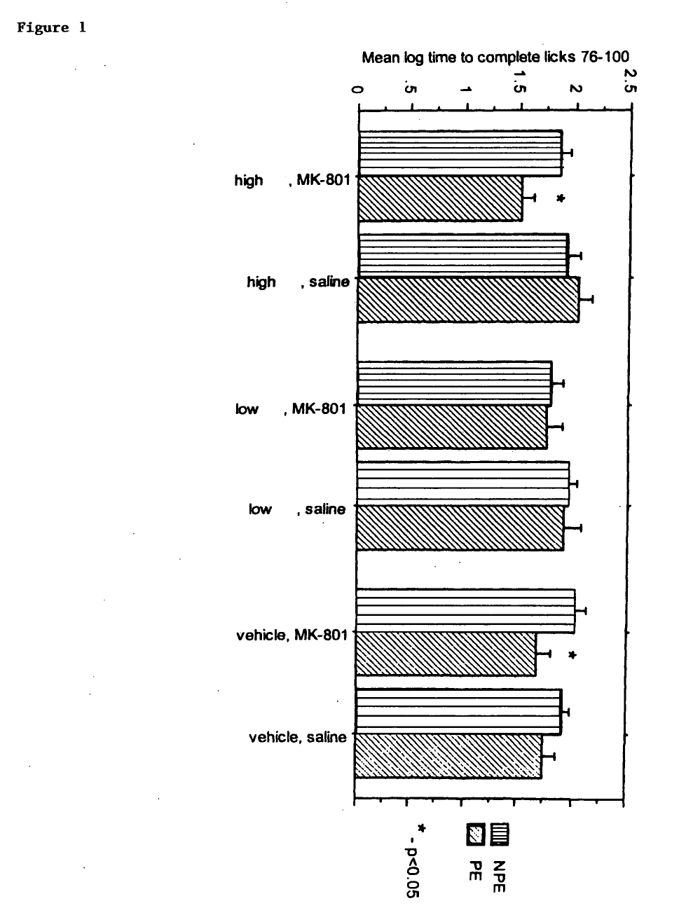

The experiment included 134 rats; data of 8 rats were dropped from the analysis. The 12 experimental groups did not differ in their times to complete licks 51-75 before tone onset (all p's>0.5; overall mean A period=10.58 sec). FIG. 1 presents the mean log times to complete licks 76-100 (after tone onset) of the preexposed and nonpreexposed groups in the 6 drug conditions: vehicle+saline, vehicle+MK-801, 25 mg/kg ladostigil tartrate+saline, 25 mg/kg ladostigil tartrate +MK-801, 37.5 mg/kg ladostigil tartrate+saline, 37.5 mg/kg ladostigil tartrate+MK-801. Three-way ANOVA with main factors of preexposure, treatment and pretreatment, yielded significant main effects of preexposure F_{(1, 114)}=4.203, p<0.05 and treatment F_{(1, 114)}=4.359, p<0.05. Post-hoc comparisons revealed a significant difference between the preexposed and nonpreexposed groups, namely, LI, in the MK-801 condition (p<0.05), and in the 37.5 mg/kg ladostigil tartrate+MK-801 condition (p<0.05) but not in the other conditions.

With the parameters of strong conditioning used here, in both experiments LI was absent in control rats, but MK-801 treated rats persisted in showing LI (p<0.01). Physostigmine on its own had no effect at both doses used, whereas it reversed abnormally persistent LI in MK-801-treated rats at the 0.15 mg/kg dose. Likewise, ladostigil tartrate on its own had no effect at both doses used, whereas it reversed abnormally persistent LI in MK-801-treated rats at the 25 mg/kg dose.

The finding that ladostigil tartrate reversed MK-801-induced LI persistence supports the notion that enhancement of acetylcholinesterase transmission can ameliorate cognitive deficits induced by NMDAR blockade.

Symptoms of schizophrenia are today commonly divided into positive symptoms, negative symptoms, and cognitive impairments. The three way division has replaced what used to be the accepted description, namely, that negative symptoms are associated with cognitive impairments. Cognitive enhancers are much sought after remedies for schizophrenics' malady. NMDAR antagonists, including MK-801, produce in healthy humans the entire spectrum of schizophrenia symptoms, namely positive, negative and cognitive impairments. NMDAR antagonist behavioral effects in animals model negative symptoms, or cognitive impairments, or negative symptoms/cognitive impairment. Abnormally persistent LI in MK-801 treated rats can be claimed to model negative symptoms, cognitive impairments or both.

What is claimed is:

1. A method of treating a symptom of schizophrenia in a subject afflicted with schizophrenia comprising administering to the subject an amount of R(+)-6-(N-methyl, N-ethyl-carbamoyloxy)-N′-propargyl-1-aminoindan or a pharmaceutically acceptable salt thereof effective to treat the symptom of schizophrenia.

2. The method of claim 1, wherein the subject is a human being.

3. The method of claim 1, wherein the symptom of the schizophrenia is a negative symptom/cognitive impairment.

4. The method of claim 1, wherein the administration is effected orally, parenterally, rectally or transdermally.

5. The method of claim 1, wherein the method comprises administering R(+)-6-(N-methyl, N-ethyl-carbamoyloxy)-N′-propargyl-1-aminoindan.

6. The method of claim 1, wherein the method comprises administering a pharmaceutically acceptable salt of R(+)-6-(N-methyl, N-ethyl-carbamoyloxy)-N′-propargyl-1-aminoindan.

7. The method of claim 6, wherein the pharmaceutically acceptable salt of R(+)-6-(N-methyl, N-ethyl-carbamoyloxy)-N′-propargyl-1-aminoindan is ½ tartrate.

8. The method of claim 7, wherein the amount of R(+)-6-(N-methyl, N-ethyl-carbamoyloxy)-N′-propargyl-1-aminoindan ½ tartrate is in the range from 0.5 mg to 2000 mg.

9. The method of claim 8, wherein the amount of R(+)-6-(N-methyl, N-ethyl-carbamoyloxy)-N′-propargyl-1-aminoindan ½ tartrate is in the range from 25 mg to 105 mg.

10. The method of claim 9, wherein the amount of R(+)-6-(N-methyl, N-ethyl-carbamoyloxy)-N′-propargyl-1-aminoindan ½ tartrate is 25 mg.

11. The method of claim 8, wherein the amount of R(+)-6-(N-methyl, N-ethyl-carbamoyloxy)-N′-propargyl-1-aminoindan ½ tartrate is 8.9 mg.

12. The method of claim 8, wherein the salt of R(+)-6-(N-methyl, N-ethyl-carbamoyloxy)-N′-propargyl-1-aminoindan is in crystalline form.

13. The method of claim 1, wherein the R(+)-6-(N-methyl, N-ethyl-carbamoyloxy)-N′-propargyl-1-aminoindan or the pharmaceutically acceptable salt thereof is in a pharmaceutical composition which also comprises at least one pharmaceutically acceptable carrier.

14. The method of claim 13, wherein the pharmaceutical composition is in the form of a tablet, capsule, pill, powder, or granule.

15. A pharmaceutical composition comprising R(+)-6-(N-methyl, N-ethyl-carbamoyloxy)-N′-propargyl-1-aminoindan or the pharmaceutically acceptable salt thereof and an agent which treats a symptom of schizophrenia.

16. The composition of claim 15, wherein the agent is chlorpromazine, thioridazine, mesoridazine, trifluoperazine, fluphenazine, perphenazine, loxapine, molindone, thiothixene, haloperidol, pimozide, clozapine, risperidone, olanzapine, quetiapine, sertindole, or ziprasidone.

17-18. (canceled)
